SEQUENCE LISTING (20 sequences)

SEQ ID NO: 1 - protein, 461 aa, synthetic construct
  REGION 1..461: note = Synthetic Construct
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKEGGGSGGGSGGGSGGGAPDVQDCPECTLQENPFFSQPGAPILQCMGCCFSRAYPTPLRSKKTMLVQKNVTSESTCCVAKSYNRVTVMGGFKVENHTACHCSTCYYHKSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 1392 bp, synthetic construct
  misc_feature 1..1392: note = Synthetic Construct
  source 1..1392: mol_type = other DNA
gccaccatgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgccactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 3 - RNA, 36 nt, synthetic construct
  misc_feature 1..36: note = Synthetic Construct
  source 1..36: mol_type = other RNA
acggttcagccatccagaatgttttagagctagaaa

SEQ ID NO: 4 - DNA, 18 bp, synthetic construct
  misc_feature 1..18: note = Synthetic Construct
  source 1..18: mol_type = other DNA
taatacgactcactatag

SEQ ID NO: 5 - DNA, 20 bp, synthetic construct
  misc_feature 1..20: note = Synthetic Construct
  source 1..20: mol_type = other DNA
gggactgagacaaaaagtaa

SEQ ID NO: 6 - DNA, 155 bp, synthetic construct
  misc_feature 1..155: note = Synthetic Construct
  source 1..155: mol_type = other DNA
taatacgactcactatagacggttcagccatccagaatgttttagagctagaaatagcaagttaaaataaggctagtccgttatcaacttgaaaaagtggcaccgagtcggtgctttttttctagacccagctttcttgtacaaagttggcatta

SEQ ID NO: 7 - DNA, 28 bp, synthetic construct
  misc_feature 1..28: note = Synthetic Construct
  source 1..28: mol_type = other DNA
taatacgactcactatagacggttcagc

SEQ ID NO: 8 - DNA, 24 bp, synthetic construct
  misc_feature 1..24: note = Synthetic Construct
  source 1..24: mol_type = other DNA
taatgccaactttgtacaagaaag

SEQ ID NO: 9 - RNA, 36 nt, synthetic construct; identical to SEQ ID NO: 3
  misc_feature 1..36: note = Synthetic Construct
  source 1..36: mol_type = other RNA

SEQ ID NO: 10 - DNA, 23 bp, synthetic construct
  misc_feature 1..23: note = Synthetic Construct
  source 1..23: mol_type = other DNA
ctgaccaacatcacaatcgccat

SEQ ID NO: 11 - DNA, 23 bp, synthetic construct
  misc_feature 1..23: note = Synthetic Construct
  source 1..23: mol_type = other DNA
gttttctggatcttgggtctggc

SEQ ID NO: 12 - DNA, 20 bp, synthetic construct
  misc_feature 1..20: note = Synthetic Construct
  source 1..20: mol_type = other DNA
acagagtgaccgtgatgggc

SEQ ID NO: 13 - DNA, 20 bp, synthetic construct
  misc_feature 1..20: note = Synthetic Construct
  source 1..20: mol_type = other DNA
ctgggaggcgattgtagggg

SEQ ID NO: 14 - DNA, 300 bp, synthetic construct
  misc_feature 1..300: note = Synthetic Construct
  source 1..300: mol_type = other DNA
gtaatatatttaaaacaatactactgaaacttgaattgggaatgacagtttcagacccgaaactaggggcatggcccactaatgaggggataagttgagtgaaagaaaatgacaactgtttacagattttggcaacatttaaaccaaggcccttctccttatgcacaacaactgctttaacagctgctttttttttctccccccctccctccccccatcttggaaatccttgtatcaggtttttgagtcatgatgcaagaatctgggactgagacaaaaagtaacggttcagccatccag

SEQ ID NO: 15 - DNA, 57 bp, synthetic construct
  misc_feature 1..57: note = Synthetic Construct
  source 1..57: mol_type = other DNA
gccacgaacttctccctgttaaagcaagcaggagacgtggaagaaaaccccggtccc

SEQ ID NO: 16 - DNA, 1386 bp, synthetic construct
  misc_feature 1..1386: note = Synthetic Construct
  source 1..1386: mol_type = other DNA
atgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgtcactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 17 - DNA, 299 bp, synthetic construct
  misc_feature 1..299: note = Synthetic Construct
  source 1..299: mol_type = other DNA
aatgggtcgggcggcagcaaccacttactagagtgcggcggtcttcgggaggggcggtccaacggagagacgccggccgtggacatcggggcagctgacctcgcccacgcccagcagcagcagcaacaggtactgggctttggagtcttgggaggtggaggtggtgggtgcgaattttactgctgaattttacacttaatcgcgatctcgcgatcagtgttttgtgttcacctgtctgtccatctgtctttctgtctgtcagtggcatctcataaaccatcagccctctaggagtccca

SEQ ID NO: 18 - DNA, 1494 bp, synthetic construct
  misc_feature 1..1494: note = Synthetic Construct
  source 1..1494: mol_type = other DNA
atgatgcaagaatctgggactgagacaaaaagtaacggttcagccatccaggccacgaacttctccctgttaaagcaagcaggagacgtggaagaaaaccccggtcccatgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgtcactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 19 - protein, 497 aa, synthetic construct
  REGION 1..497: note = Synthetic Construct
MMQESGTETKSNGSAIQATNFSLLKQAGDVEENPGPMKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKEGGGSGGGSGGGSGGGAPDVQDCPECTLQENPFFSQPGAPILQCMGCCFSRAYPTPLRSKKTMLVQKNVTSESTCCVAKSYNRVTVMGGFKVENHTACHCSTCYYHKSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 2043 bp, synthetic construct
  misc_feature 1..2043: note = Synthetic Construct
  source 1..2043: mol_type = other DNA
gtaatatatttaaaacaatactactgaaacttgaattgggaatgacagtttcagacccgaaactaggggcatggcccactaatgaggggataagttgagtgaaagaaaatgacaactgtttacagattttggcaacatttaaaccaaggcccttctccttatgcacaacaactgctttaacagctgctttttttttctccccccctccctccccccatcttggaaatccttgtatcaggtttttgagtcatgatgcaagaatctgggactgagacaaaaagtaacggttcagccatccaggccacgaacttctccctgttaaagcaagcaggagacgtggaagaaaaccccggtcccatgaagaccctgcagttcttcttcctgttctgctgctggaaggccatctgctgcaacagctgcgagctgaccaacatcacaatcgccatcgagaaagaggaatgccggttctgcatcagcatcaacaccacttggtgcgccggctactgctacacccgggacctggtgtacaaggaccccgccagacccaagatccagaaaacctgcaccttcaaagaactggtgtacgagacagtgcgggtgcccggatgtgcccaccatgccgatagcctgtacacctaccctgtggccacccagtgtcactgcggcaagtgcgatagcgacagcaccgattgcaccgtgcggggactgggccctagctactgtagcttcggcgagatgaaggaaggcggcggatctggcggaggaagcggagggggatctgggggcggagcacctgatgtgcaggattgccctgagtgcaccctgcaggaaaacccattcttcagccagcctggcgcccctatcctgcagtgcatgggctgctgcttcagcagagcctaccccacccccctgcggagcaagaaaaccatgctggtgcagaaaaacgtgaccagcgagagcacctgttgcgtggccaagagctacaacagagtgaccgtgatgggcggcttcaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataaaatgggtcgggcggcagcaaccacttactagagtgcggcggtcttcgggaggggcggtccaacggagagacgccggccgtggacatcggggcagctgacctcgcccacgcccagcagcagcagcaacaggtactgggctttggagtcttgggaggtggaggtggtgggtgcgaattttactgctgaattttacacttaatcgcgatctcgcgatcagtgttttgtgttcacctgtctgtccatctgtctttctgtctgtcagtggcatctcataaaccatcagccctctaggagtcccag

(19) United States
(12) Patent Application Publication
(10) Pub. No.: US 2025/0179184 A1
(43) Pub. Date: Jun. 5, 2025

(54) FOXP1-ABLATED CHIMERIC CELLS

(71) Applicant: H. Lee Moffitt Cancer Center and Research Institute, Inc., Tampa, FL (US)

(72) Inventor: Jose Conejo-Garcia, Tampa, FL (US)

(21) Appl. No.: 19/054,136

(22) Filed: Feb. 14, 2025

Related U.S. Application Data
(63) Continuation of application No. 17/297,897, filed on May 27, 2021.
(60) Provisional application No. 62/789,061, filed on Jan. 7, 2019.

(51) Int. Cl. C07K 16/28; A61K 40/11; A61K 40/31; A61K 40/42

(52) U.S. Cl. CPC C07K 16/2818; A61K 40/11; A61K 40/31; A61K 40/423; A61K 40/4237; A61K 40/4242; C07K 16/2827; A61K 2239/31; A61K 2239/59

(57) ABSTRACT

Disclosed herein are non-viral methods to ablate FOXP1 in T cells while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous FOXP1 protein.

### CROSS-REFERENCE TO RELATED APPLICATIONS

This application is a continuation of U.S. patent application Ser. No. 17/297,897, filed May 27, 2021, which is a National Stage of International Application No. PCT/US2020/012319, filed Jan. 6, 2020, which claims benefit of U.S. Provisional Application No. 62/789,061, filed Jan. 7, 2019, which are hereby incorporated herein by reference in their entirety.

### SEQUENCE LISTING

This application contains a sequence listing filed in ST.26 format entitled “320803-1841 Sequence Listing” created on Feb. 10, 2025, and having 34,071 bytes. The content of the sequence listing is incorporated herein in its entirety.

### BACKGROUND

Epithelial ovarian cancer is one of the deadliest tumors, killing >14,000 women each year in the United States (Jemal, A., et al. CA Cancer J Clin 2009 59: 225-249). Ovarian cancer is, however, an immunogenic tumor, as our group has contributed to establish in the last decade (Curiel, T. J., et al. Nat Med 2004 10: 942-949; Zhang, L., et al. N Engl J Med 2003 348: 203-213; Cubillos-Ruiz, J. R., et al. J Clin Invest 2009 119: 2231-2244; Cubillos-Ruiz, J. R., et al. Oncotarget 2010 1: 329-328; Huarte, E., et al. Cancer Res 2008 68: 7684-7691; Nesbeth, Y., et al. Cancer Res 2009 69: 6331-6338; Scarlett, U. K., et al. Cancer Res 2009 69: 7329-7337; Scarlett, U. K., et al. J Exp Med 2012 209: 495-506; Cubillos-Ruiz, J. R., et al. Cancer Res 2012 72: 1683-1693). Immunotherapies therefore offer great promise to reverse this dismal prognosis.

In recent years, transferring autologous T cells engineered to express chimeric antigen receptors (CARs) has shown impressive cures for patients with hematologic malignancies (Porter, D. L., et al. N Engl J Med 2011 365: 725-733; Maus, M. V., et al. Blood 2014 123: 2625-2635; Kalos, M., et al. Sci Transl Med 2011 3: 95ra73). Several hurdles, however, have so far prevented the success of CAR T cells against solid tumors, including ovarian cancer. Thus, epithelial malignancies orchestrate a hostile environment where immunosuppressive networks converge to abrogate T cell effector activity (Zou, W. Nat Rev Cancer 2005 5: 263-274).

### SUMMARY

As disclosed herein, there is a common targetable mechanism of T cell unresponsiveness in cancer driven by the upregulation of the transcription factor Forkhead box protein P1 (FOXP1), which prevents CD8⁺ T cells from proliferating and upregulating Granzyme-B and interferon-γ in response to tumor antigens. Non-viral methods were developed to ablate Forkhead box protein P1 (FoxP1) in T cells while effectively expressing chimeric receptors re-directing their effector activity against FSHR+ ovarian cancer cells. T cell unresponsiveness in cancer is partially driven by the upregulation of the transcription factor Foxp1, which mediated the suppressive effects of TGF-β, preventing CD8⁺ T cells from proliferating and upregulating Granzyme-B and interferon-γ in response to tumor antigens. Preliminary results showed that ablation of FOXP1 in CD8⁺ T cells allowed proliferation and upregulation of Granzyme-B and interferon-γ in response to tumor antigens. In addition, Foxp1-deficient lymphocytes induced rejection of incurable tumors and promoted protection against tumor re-challenge. Further, consistent with the superior effector activity of Foxp1-deficient TILs, adoptive transfer of tumor antigen-primed CTLs dramatically delayed the progression of established and aggressive orthotopic tumors, while identically activated control T-cells only induced modest protection.

Therefore, disclosed herein are non-viral methods to ablate FOXP1 in T cells while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous FOXP1 protein.

FOXP1.1 is constitutively expressed in human T cells. Therefore, in some embodiments, the transgene is inserted into a Foxp1 gene (such as Foxp1.1) loci, thereby disrupting gene transcription and ensuring constitutive expression of the transgene. The transgene can be inserted at any loci within the Foxp1 gene that would disrupt gene transcription while also allowing for transcription of the transgene. For example, in some cases, the transgene is inserted into exon 1 of FoxP1.1.

Additional genes that is deleterious to T cells and constitutively active include CTLA4, CBLB, CD5, TMEM222, SOCS1, GNA13, CDKN1B, RNF7, and PTPN. Therefore, in some embodiments, the transgene is inserted into a CTLA4, CBLB, CD5, TMEM222, SOCS1, GNA13, CDKN1B, RNF7, or PTPN gene loci, thereby disrupting gene transcription and ensuring constitutive expression of the transgene. Additional negative regulators of T cell responses include TCEB2, CUL5, UBASH3A, DGKZ, DGKA, TNFAIP3, NDUFB10, FIBP, RASA2, SMARCB1, PCBP2, ARIH2, ZFP36L1, MEF2D, AGO1, and RPRD1B. Therefore, in some embodiments, the disclosed transgene is inserted into a TCEB2, CUL5, UBASH3A, DGKZ, DGKA, TNFAIP3, NDUFB10, FIBP, RASA2, SMARCB1, PCBP2, ARIH2, ZFP36L1, MEF2D, AGO1, or RPRD1B loci.

Site-specific insertion of the transgene can be done, for example, by gene editing techniques, such as CRISPR or TALEN. In some cases, 2 different gene editing systems are used: one for integration of the transgene, and another one for effective ablation of all FoxP1 variants (for instance, with a target at exon 10, common to all of them).

In some embodiments, the chimeric receptor comprises a chimeric antigen receptor (CAR) polypeptide. CARs generally combine an antigen recognition domain with transmembrane signaling motifs involved in lymphocyte activation. The antigen recognition domain can be, for example, the single-chain variable fragments (scFv) of a monoclonal antibody (mAb) or a fragment of a natural ligand that binds a target receptor. CARs are generally made up of three domains: an ectodomain, a transmembrane domain, and an endodomain. The ectodomain comprises the antigen recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

In some embodiments, the chimeric receptor comprises two subunits of a follicule-stimulating hormone (FSH), which binds FSH receptors. These chimeric receptors are referenced to herein as “chimeric endocrine receptors (CERs)” and can target FSH-positive ovarian tumors. In some embodiments, the CER contains two subunits of FSH (FSHβ and CGα), separated by a linker.

In some embodiments, the chimeric receptor has the following structure: Signal Peptide (FSH beta)_hFSH beta_Spacer_hFSH alpha_Hinge (from human CD8)_TM domain (from human CD8)_human 4-1BB (intracellular)_human CD3z domain.

In some embodiments, the chimeric receptor has the amino acid sequence: MKTLQFFFLFCCWKAICC_NSCELTNITIAIEKEECRFCISINTTWCAGYCYTRDLVYKDPAR PKIQKTCTFKELVYETVRVPGCAHHADSLYTYPVATQCHCGKCDSDSTDCTVRGLGPSYC SFGEMKE_GGGSGGGSGGGSGGG_APDVQDCPECTLQENPFFSQPGAPILQCMGCCFS RAYPTPLRSKKTMLVQKNVTSESTCCVAKSYNRVTVMGGFKVENHTACHCSTCYYHK_SA STTTPAPRPPTPAPTIASQPLSLRPEACRPAAGGAVHTRGLDFACD_IYIWAPLAGTCGVLLL SLVITLYC_KRGRKKLLYIFKQPFMRPVQTTQEEDGCSCRFPEEEEGGCEL_RVKFSRSADA PAYKQGQNQLYNELNLGRREEYDVLDKRRGRDPEMGGKPRRKNPQEGLYNELQKDKMA EAYSEIGMKGERRRGKGHDGLYQGLSTATKDTYDALHMQALPPR (SEQ ID NO:1), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:1.

Therefore, in some embodiments, the chimeric receptor is encoded by the following nucleic acid sequence:

(SEQ ID NO: 2)
GCCACCATGAAGACCCTGCAGTTCTTCTTCCTGTTCTGCTGCTGGAAGG
CCATCTGCTGCAACAGCTGCGAGCTGACCAACATCACAATCGCCATCGA
GAAAGAGGAATGCCGGTTCTGCATCAGCATCAACACCACTTGGTGCGCC
GGCTACTGCTACACCCGGGACCTGGTGTACAAGGACCCCGCCAGACCCA
AGATCCAGAAAACCTGCACCTTCAAAGAACTGGTGTACGAGACAGTGCG
GGTGCCCGGATGTGCCCACCATGCCGATAGCCTGTACACCTACCCTGTG
GCCACCCAGTGCCACTGCGGCAAGTGCGATAGCGACAGCACCGATTGCA
CCGTGCGGGGACTGGGCCCTAGCTACTGTAGCTTCGGCGAGATGAAGGA
AGGCGGCGGATCTGGCGGAGGAAGCGGAGGGGGATCTGGGGGCGGAGCA
CCTGATGTGCAGGATTGCCCTGAGTGCACCCTGCAGGAAAACCCATTCT
TCAGCCAGCCTGGCGCCCCTATCCTGCAGTGCATGGGCTGCTGCTTCAG
CAGAGCCTACCCCACCCCCCTGCGGAGCAAGAAAACCATGCTGGTGCAG
AAAAACGTGACCAGCGAGAGCACCTGTTGCGTGGCCAAGAGCTACAACA
GAGTGACCGTGATGGGCGGCTTCAAGGTGGAAAACCACACCGCCTGCCA
CTGCAGCACATGCTACTACCACAAGAGCGCTAGCACCACCACCCCTGCC
CCTAGACCTCCAACACCCGCCCCTACAATCGCCTCCCAGCCTCTGTCTC
TGAGGCCCGAGGCTTGTAGACCAGCTGCTGGCGGAGCCGTGCACACCAG
AGGACTGGATTTCGCCTGCGACATCTACATCTGGGCCCCTCTGGCCGGC
ACATGTGGCGTGCTGCTGCTGAGCCTCGTGATCACCCTGTACTGCAAGC
GGGGCAGAAAGAAGCTGCTGTACATCTTCAAGCAGCCCTTCATGCGGCC
CGTGCAGACCACCCAGGAAGAGGACGGCTGCTCCTGCAGATTCCCCGAA
GAGGAAGAGGGGGGCTGCGAACTGAGAGTGAAGTTCAGCAGAAGCGCCG
ACGCCCCTGCCTACAAGCAGGGCCAGAACCAGCTGTACAACGAGCTGAA
CCTGGGCAGACGGGAAGAGTACGACGTGCTGGACAAGCGGAGAGGCAGG
GACCCTGAGATGGGCGGAAAGCCCAGACGGAAGAACCCCCAGGAAGGCC
TGTATAACGAACTGCAGAAAGACAAGATGGCCGAGGCCTACAGCGAGAT
CGGAATGAAGGGCGAGCGGAGAAGAGGCAAGGGCCACGATGGCCTGTAC
CAGGGCCTGAGCACCGCCACCAAGGACACCTATGACGCCCTGCACATGC
AGGCCCTGCCCCCCAGATAA.

Also disclosed are isolated nucleic acid sequences encoding the disclosed polypeptides, vectors comprising these isolated nucleic acids, and cells containing these vectors. The chimeric cells disclosed herein can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the cell exhibits an anti-tumor immunity when the antigen binding domain of the CAR binds to CD123.

Also disclosed is a method of providing an anti-cancer immunity in a subject, comprising administering to the subject an effective amount of a chimeric cell disclosed herein, thereby providing an anti-tumor immunity in the subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C show Foxp1 expression regulates anti-tumor effector functions and proliferation of CD8-T cells in the TME. FIG. 1A shows absolute cell number of these lymphocytes recovered from peritoneal wash 7 days after adoptive transfer into day 24 ID8-Defb29/Vegf-a tumor-bearing congenic mice. Representative of four independent experiments (p<0.05, Student's t-test). FIG. 1B shows in vivo proliferation of tumor-reactive Foxp1-deficient vs. wild-type CD8 T-cells. Lymphocytes primed for 7 d against tumor antigens were labeled with cell trace violet, adoptively transferred into orthotopic advanced ovarian cancer-bearing congenic mice, and recovered from peritoneal wash 3 and 8 days later. Representative of three experiments. FIG. 1C shows IFN-γ and Granzyme B ELISPOT analysis of T-cells primed against tumor antigens as above for 7 days.

FIGS. 2A to 2B show Foxp1 expression impairs the protective function of tumor-reactive T-cells. FIG. 2A shows on day 24 after ID8-Defb29/Vegf-a tumor challenge, 47 different CD45.1+ mice received 106 tumor antigen-primed (day 7) T-cells from Foxp1-deficient (n=16) or wild-type (n=15) CD45.2+ mice. Sixteen additional control tumor-bearing mice were treated with PBS. Data pooled from three independent experiments. P<0.0001, Mantel-Cox test. FIG. 2B shows four tumor-bearing mice surviving >60 d. after treatment with Foxp1^{-/-} T cells and six control age-matched wild-type mice were re-challenged with 2×10⁶ ID8-Defb29/Vegf-a tumor cells, administered into the axillary flank. Tumor growth was monitored in three independent experiments. P<0.003, Student's t-test.

FIGS. 3A and 3B show ovarian cancer TRM CD8+ T cells show markers of enhanced effector activity and exhaustion. FIG. 3A is a heatmap of differential expression of transcriptions factors (RBPJ), effector molecules (GZMB, IFNG), TRM determinants (CD103/ITGAE) and exhaustion markers (PD-1/PDCD1, TIM3/HAVCR2, CTLA4 and LAG3) in CD8+CD103+CD69+ vs. CD8+CD103− T cells FACS sorted from the same tumors. FIG. 3B shows clonality index (Adaptive Biotech) of TRM vs. re-circulating CD8+ T cells sorted viable single cell suspensions from 8 different freshly dissociated human serous ovarian carcinomas (stage III/IV). Gated on Zombie Yellowneg (viable) cells. *P<0.05, Mann-Whitney).

FIG. 4 illustrates FSHCAR ready for expression in T cells.

FIGS. 5A to 5C show human FSH CAR T cells kill ovarian tumor cells in a dose-dependent manner. FIG. 5A and 5B show cytotoxicity of FSHCER or mock transduced T-cells of OVCAR-3 and K562 measured by 7-AAD/Annexin V flow cytometric staining after co-culture of 18 hours. Human T cells were expanded with CD3/CD28 beads, spininfected with hFSHCAR in pBMN with retronectin or mock-transduced at 20 and 44 hours, and kept at 0.5-1 million cells/mL with 1 μg/mL of IL-7 and 20 U/mL of IL-2. At day 7, CAR and control T cells were sorted on GFP expression and rested for 18 hours, before being plated with plated with human OVCAR-3 ovarian cancer cells (10000 per well; spontaneously FSHR+) on the indicated effector (E) to target (T) ratios. Six hours after setting the coculture cells were stained with Annexin V and 7AAD and cytotoxicity was analyzed by flow cytometry. The percentage of specific lysis was calculated as (experimental dead−spontaneous dead)/(maximum dead−spontaneous dead)×100%. FIG. 5C show tumor volume of an ovarian patient-derived xenograft tumor grown in the flank of NOD-SCID mice (n=2 mice per tumor, one case-one control) injected intratumorally with 10 million FSHCER or mock transduced T-cells (arrows mark time of T-cell injection).

FIG. 6 shows FSH-CERT cells abrogate the progression of FSHR-expressing orthotopic ovarian tumors. T cells carrying FSHR-targeting CERs (FSH-CER) or identically expanded mock-transduced T cells (pBMN) were intraperitoneally administered at days 7 and 14 after intraperitoneal challenge with ID8-Defb29/Vegf-a tumor cells transduced with FSHR. Malignant progression was compared. Representative of 2 independent experiments (10 mice/group).

FIG. 7 shows advanced human ovarian carcinoma specimens express variable levels of FSHR. FSHR protein expression was analyzed by WB (Santa Cruz #H-190) in 6 unselected human advanced ovarian carcinoma specimens, and compared to that in FSH-targeted CAR T cell-sensitive OVCAR3 cells. β-actin Ab, Sigma #A5441.

FIG. 8 shows constitutive robust expression of FOXP1 in human T cells. FOXP1 was detected by WB in lysates from the indicated (viable) T cell subsets sorted from the blood of healthy donors or single-cell suspensions from 2 different freshly dissociated serous advanced ovarian carcinomas. The membrane was stained with anti-Foxp1 Abs (rabbit polyclonal; AAs 685-705; Abmart; 1:1000 dilution).

FIG. 9 illustrates a scheme of the HDR template used to replace FOXP1 with P2A-FSHCER cassette.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### FoxP1 Disruption

Disclosed herein are non-viral methods to ablate FOXP1 in T cells while effectively expressing chimeric receptors. Therefore, disclosed herein is a chimeric cell expressing a chimeric receptor, wherein the chimeric receptor is encoded by a transgene, and wherein the transgene is inserted in the genome of the cell at a location that disrupts expression or activity of an endogenous FOXP1 protein.

In some embodiments, the transgene is inserted into the Foxp1 gene loci, thereby disrupting gene transcription. The transgene can be inserted at any loci within the Foxp1 gene that would disrupt gene transcription.

Site-specific insertion of the transgene can be done, for example, by gene editing techniques, such as CRISPR.

### Chimeric Endocrine Receptors (CERs)

In some embodiments, the chimeric receptor comprises two subunits of a follicule-stimulating hormone (FSH), which binds FSH receptors. These chimeric receptors are referenced to herein as “chimeric endocrine receptors (CERs)” and can target FSH-positive ovarian tumors. In some embodiments, the CER contains two subunits of FSH (FSHB and CGα), separated by a linker.

### Chimeric Antigen Receptors (CAR)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3: 35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16(9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or SCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18: 676-84; Maher J, et al. Nat Biotechnol 2002 20: 70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGAL, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in

WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors. Also disclosed are oligonucleotides for use in inserting the chimeric receptors into the genome of a T cell at a site that will disrupt Foxp1 expression or activity.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Immune Effector Cells

Also disclosed are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53: 1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23: 427-431). NK cells are safer effector cells, as they may avoid the potentially lethal complications of cytokine storms (Morgan R A, et al. Mol Ther 2010 18: 843-851), tumor lysis syndrome (Porter D L, et al. N Engl J Med 2011 365: 725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53: 1666-1676; Fauriat C, et al. Leukemia 2006 20: 732-733), the means by which one might enhance NK cell-mediated anti-MM activity has been largely unexplored prior to the disclosed CARs.

### Therapeutic Methods

Immune effector cells expressing the disclosed chimeric receptors can elicit an anti-tumor immune response against cancer cells. The anti-tumor immune response elicited by the disclosed chimeric cells may be an active or a passive immune response. In addition, the immune response may be part of an adoptive immunotherapy approach in which chimeric cells induce an immune response specific to the target antigen.

Adoptive transfer of immune effector cells expressing chimeric receptors is a promising anti-cancer therapeutic. Following the collection of a patient's immune effector cells, the cells may be genetically engineered to express the disclosed chimeric receptors while ablating Foxp1 according to the disclosed methods, then infused back into the patient.

The disclosed chimeric effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed chimeric cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed chimeric cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed chimeric cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed chimeric cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with chimeric cells for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed chimeric cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Foxp1 Overexpression Prevents Tumor-Reactive T-Cells from Proliferating in the TME

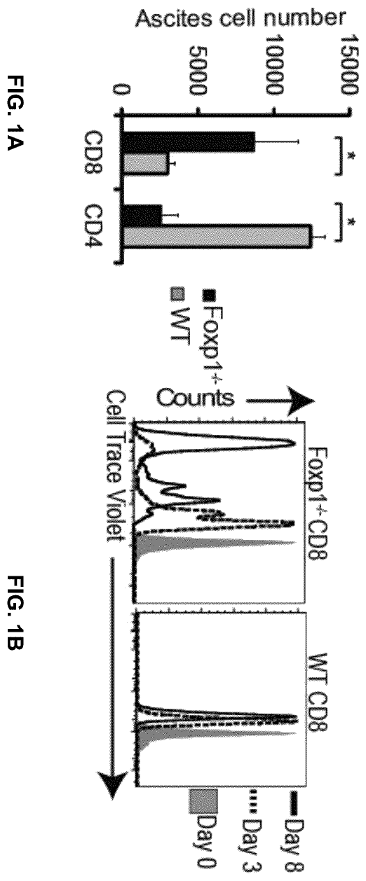

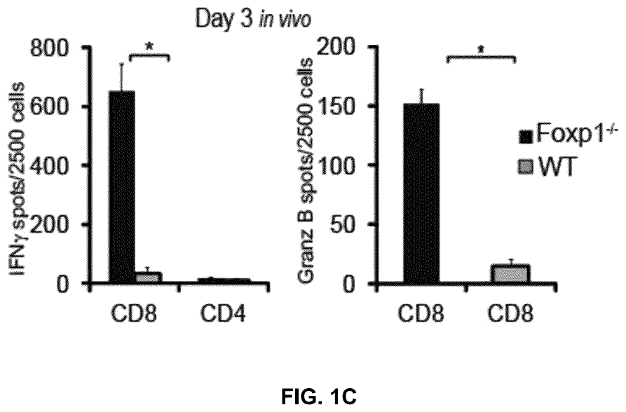

Foxp1 is overexpressed in mouse and human anti-tumor T cells. To elucidate its biological role, naïve Foxp1-deficient T cells (from CD4^{Cre}Foxp1^{f/f} mice (Stephen, T. L., et al. Immunity 2014 41: 427-439)) and T cells from Foxp1f/f littermates with ID8-Defb29/Vegf-a-tumor (Cubillos-Ruiz, J. R., et al. J Clin Invest 2009 119: 2231-2244; Scarlett, U. K., et al. Cancer Res 2009 69: 7329-7337; Scarlett, U. K., et al. J Exp Med 2012 209: 495-506; Conejo-Garcia, J. R., et al. Nat Med 2004 10: 950-958; Cubillos-Ruiz, J. R., et al. Cell 2015 161: 1527-1538; Stephen, T. L., et al. Immunity 2017 46:51-64) pulsed BMDCs were primed, a method that drives the enrichment of tumor-reactive T cells (Nesbeth, Y., et al. Cancer Res 2009 69: 6331-6338; Stephen, T. L., et al. Immunity 2014 41: 427-439; Nesbeth, Y. C., et al. J Immunol 2010 184: 5654-5662). Ex vivo stimulation resulted in comparable CD8/CD4 ratios in Foxp1⁺ and Foxp1^{-/-} lymphocytes. However, when equal numbers of Foxp1-deficient and control tumor-reactive T-cells were adoptively transferred into i.p. ID8-Defb29/Vegf-a-tumor-bearing, congenic mice, the proportions and absolute numbers of CD8 T-cells lacking Foxp1 were ˜4-fold increased (FIG. 1A). Foxp1^{-/-} CD8 T-cells expanded in vivo at tumor beds, as shown by dilution of Cell Trace Violet by Foxp1-deficient, but not control CD8 T-cells (FIG. 1B). Foxp1 overexpressing T-cells did not undergo significant death, but remained unresponsive at tumor beds. In addition, significantly higher numbers of Foxp1-deficient T-cells sorted from peritoneal wash after 3 days in the TME reacted by secreting IFN-γ and Granzyme-B in re-call ELISPOT analysis, compared to identically handled FoxP1⁺ CD8 lymphocytes (FIG. 1C). Therefore, Foxp1 ablation licenses tumor-reactive T-cells to expand at tumor beds in response to antigen and exert their anti-tumor activity.

### Example 2: Foxp1 Ablation Empowers Tumor-Reactive T Cells to Control Incurable Tumors

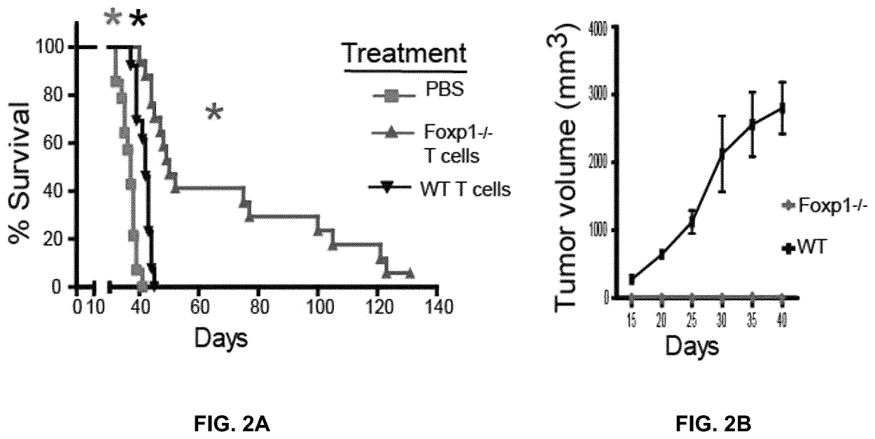

Consistent with the superior effector activity of Foxp1-deficient TILs, adoptive transfer of tumor antigen-primed CTLs from CD4^{Cre}Foxp1^{f/f} mice dramatically delayed the progression of established and aggressive ID8-Defb29/Vegf-a orthotopic tumors, while identically activated control T-cells only induced modest protection (FIG. 2A). Foxp1^{-/-} T cells primed against 3T3-pulsed BMDCs showed no effect, confirming antigen specificity. Notably, a fraction of mice treated with Foxp1-deficient T-cells in every independent experiment never showed signs of disease. To define whether Foxp1-deficient lymphocytes promoted long-term protection against tumor recurrences, these mice were re-challenged with ID8-Defb29/Vegf-a flank (axillary) tumors. As shown in FIG. 2B, all long-term survivors rejected secondary tumors, while all control (naïve) mice developed >2 cm tumors. Therefore, Foxp1 ablation multiplies the anti-tumor activity of anti-tumor cytotoxic T-cells in the TME.

### Example 3: TRM Ovarian Cancer TILs Exhibit Higher Levels of Effector Mediators and Exhaustion Markers

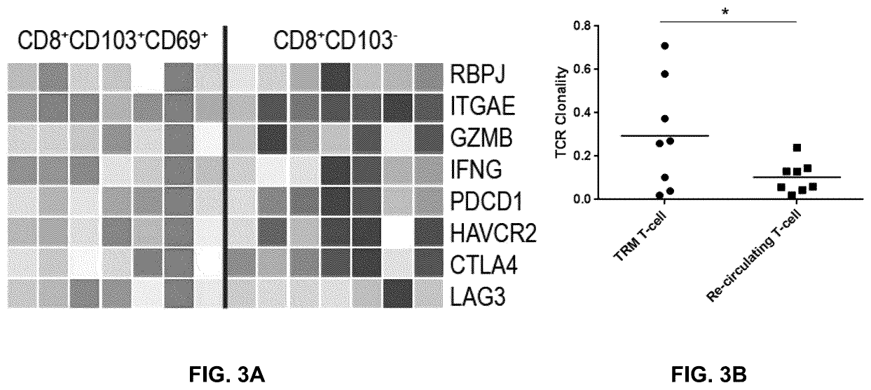

As aforementioned, cytolytic responses in NSCLC appear to be governed by a population of TRM cells, the density of which is also associated with better prognosis in human ovarian cancer (Webb, J. R., et al. Clin Cancer Res 2014 20: 434-444). To better understand the role of TRM cells in protective responses specifically against ovarian cancer, CD8⁺CD103⁺CD69⁺ TRM and CD8⁺CD103⁻ cells were FACS-sorted from viable single cell-suspensions generated from 7 different freshly dissociated human advanced serous ovarian carcinomas (Cubillos-Ruiz, J. R., et al. J Clin Invest 2009 119: 2231-2244; Cubillos-Ruiz, J. R., et al. Oncotarget 2010 1: 329-328; Scarlett, U. K., et al. Cancer Res 2009 69: 7329-7337; Scarlett, U. K., et al. J Exp Med 2012 209: 495-506; Stephen, T. L., et al. Immunity 2014 41: 427-439; Perales-Puchalt, A., et al. Clin Cancer Res 2017 23: 441-453; Stephen, T. L., et al. Immunity 2017 46: 51-64), which were then used to perform RNA-seq. Supporting previous reports in other diseases (Webb, J. R., et al. Clin Cancer Res 2014 20: 434-444), TRM (˜60% of total CD8+ TILs) showed significantly higher levels of drivers of cytolytic activity (GZMB) or other effector mediators (IFNG), but also overexpressed the classical exhaustion/activation markers PD-1, LAG3, TIM3 or CTLA4 (FIG. 3A). More unexpectedly, analysis of the TCR repertoire of 8 tumor samples through ImmunoSeq (Adaptive) showed very limited overlap of T cell specificities between TRM vs. re-circulating CD8+ T cells sorted from the same tumor (ranging from 6% to 20%; average, 11%). Although there were significant differences between patients, the clonality index of TRM CD8+ T cells was significantly higher than that of their CD103− counterparts, suggesting an enrichment for antigen-specific populations in the former (FIG. 3B). Together, these data support that anti-tumor immune pressure in ovarian cancer is exerted by a distinct population of TRM T cells that, despite obvious signs of exhaustion, exhibits higher levels of effector mediators and clonality.

### Example 4: FOXP1 Targets the Transcriptional Program Leading to TRM Differentiation

FoxP1^{-/-} tumor-reactive T cells accumulate at tumor beds and constitutively express CD69 (Stephen, T. L., et al. Immunity 2014 41: 427-439), a marker of TRM cells (Mami-Chouaib, F., et al. J Immunother Cancer 2018 6: 87). Because TRM cells appear to be responsible for anti-tumor immune pressure in ovarian cancer, the next goal was to understand how the repressive activity (Stephen, T. L., et al. Immunity 2014 41: 427-439) of FOXP1 could target the expression of genes involved in the differentiation or activity of TRM cells. For that purpose, ChlP-seq experiments were conducted using mouse CD8⁺ T cell splenocytes and custom antibodies, or control IgG. As shown in Table 1, the promoter of the transcription factor RUNX3, which programs CD8+ T cell residency in tumors (Milner, J. J., et al. Nature 2017 552: 253-257), is heavily occupied by FoxP1, along with RBPJ, which mediates crucial NOTCH signals necessary for TRM differentiation (Mami-Chouaib, F., et al. J Immunother Cancer 2018 6: 87). Furthermore, and explaining its constitutive expression in FoxP1-deficient T cells, CD69 is also target near the TSS. These results support that FOXP1-ablated CAR/CER T cells could be better equipped to acquire the features that will allow them to maintain their effector activity in established tumors.

TABLE 1
Selected promoters involved in TRM differentiation are
occupied by the transcriptional repressor FoxP1.

| Gene | Distance to TSS | FoxP1/IgG |
|---|---|---|
| CD69 | −123 | 158.1 |
| Ifngr2 | −1470 | 62.19 |
| Runx3 | −175 | 49.24 |
| Ifngr1 | −103 | 42.76 |
| Rbpj | −1399 | 38.87 |

CD8+ T cell splenocytes were crosslinked and sonicated, and DNA fragments associated with FoxP1 were selectively immunoprecipitated and sequenced. Irrelevant IgGs were identically used to establish the basal signal. Results are expressed as the center of the peak of occupancy of FoxP1, relative to the Transcription Start Site (TSS) for that particular gene, as well as the ratio between the signal using FoxP1 Abs vs. control IgGs.

### Example 5: Generation of Human and Mouse FSHR-Targeting CERs

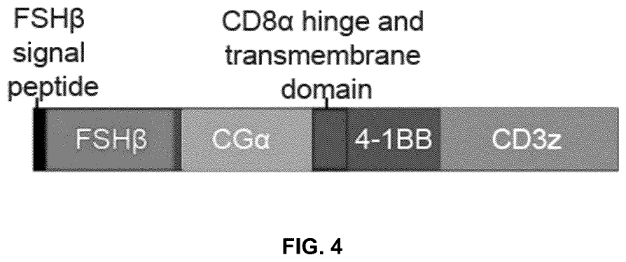

Human and mouse 4-1BB-based chimeric receptors were generated against FSHR⁺ ovarian cancer cells that includes all signals successfully used in leukemia patients (Perales-Puchalt, A., et al. Clin Cancer Res 2017 23: 441-453). To target FSHR, a construct was synthetized containing a signal peptide, followed by the two subunits of FSH (FSHβ and CGα, the latter common to LH and TSH), separated by a linker (FIG. 4). This targeting motif was cloned in frame with a hinge domain from CD8α, followed by the transmembrane domain of CD8α, the intracellular domain of co-stimulatory 4-1BB and the activating CD3ζ domain. Because engagement of FSH with it endogenous FSH Receptor is expected to occur in the absence of any antigen-antibody reaction, this construct was termed Chimeric Endocrine Receptor (CER). In parallel, CERs were generated with the corresponding mouse sequences for expression in mouse T cells.

### Example 6: Human FSH CAR T Cells Kill Ovarian Cancer Cells in a Dose-Dependent Manner

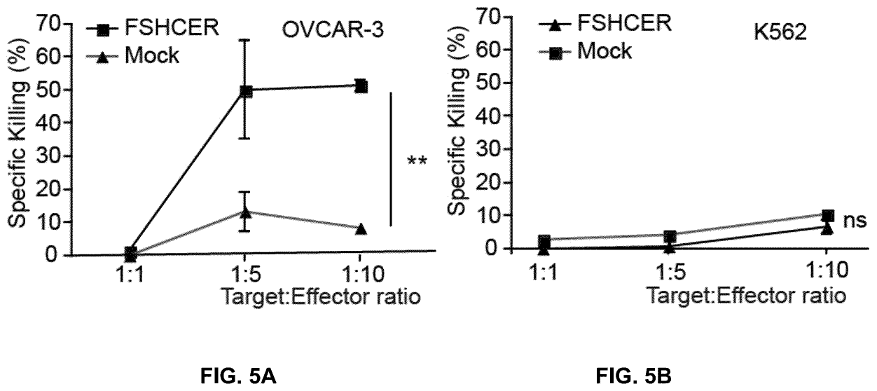

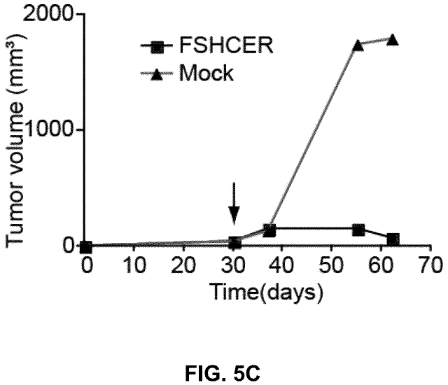

To demonstrate the activity of human FSH-CERT cells, T cells were next transduced with the human CER construct, and FSHR+ OVCAR3 (Avanzi, M. P., et al. Cell Rep 2018 23: 2130-2141; Svoronos, N., et al. Cancer Discov 2017 7: 72-85) (human ovarian cancer) cells were used as targets in a cytotoxicity experiment. As shown in FIG. 5, FSH-CERT cells effectively killed tumor cells in a dose-dependent manner. To understand the immunotherapeutic potential of targeting FSHR in clinical tumors, pairs of NSG mice growing identically established FSHR+ ovarian patient-derived xenograft (PDX) tumors were next treated with FSH-CERT cells (case mouse) or mock transduced T-cells (control paired mouse with the same xenograft). Remarkably, FSH-CER T cells induced rejection of the human ovarian PDX model expressing the highest levels of FSHR (FIG. 5C), and delayed the growth of 2 additional PDX. In both cases, administration of mock transduced T-cells into paired mice growing the same PDX tumor allowed for steady tumor growth. These data, along with specific targeting of established tumor cells in vivo (Perales-Puchalt, A., et al. Clin Cancer Res 2017 23: 441-453), support the potential of specific targeting of ovarian cancer using endogenous FSH as a targeting motif.

### Example 7: Intraperitoneal Administration of FSH-CERT Cells Delays the Progression of FSHR+ Orthotopic Ovarian Tumors in Syngeneic Mice

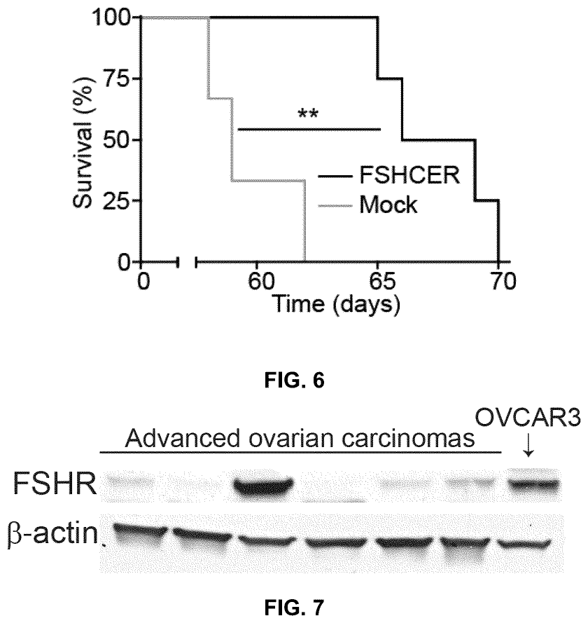

To gain some insight into the potential of administering FSH CAR T cells directly into the ovarian cancer microenvironment in syngeneic hosts, T cell splenocytes were transduced with the murine FSHCER, which targets mouse FSHR. When two cohorts of established orthotopic ovarian cancer-bearing mice were treated with only 2 injections of 1.5e6 FSH-CERT cells, all mice receiving mock-transduced T cells succumb to the disease while all the mice receiving FSH-CER lymphocytes remained alive (FIG. 6). However, in this highly ascitogenic model (Cubillos-Ruiz, J. R., et al. J Clin Invest 2009 119: 2231-2244; Scarlett, U. K., et al. J Exp Med 2012 209: 495-506; Conejo-Garcia, J. R., et al. Nat Med 2004 10: 950-958; Cubillos-Ruiz, J. R., et al. Cell 2015 161: 1527-1538; Song, M., et al. Nature 2018 562: 423-428), FSH-CERT cells were not able to eliminate tumors. Importantly, FSHCERT cells remained in the peritoneal cavity at terminal disease (Perales-Puchalt, A., et al. Clin Cancer Res 2017 23: 441-453), indicating that the TME abrogates their effector activity.

### Example 8: Most Human Ovarian Cancers Express FSHR

Independent publications support that 50-70% of ovarian cancers express the FSHR (Zhang, X. Y., et al. Cancer Res 2009 69: 6506-6514; Al-Timimi, A., et al. Br J Cancer 1986 53: 321-329; Zhang, X., et al. Int J Pharm 2013 453: 498-505; Minegishi, T., et al. Clin Cancer Res 2000 6: 2764-2770; Nakano, R., et al. Am J Obstet Gynecol 1989 161: 905-910; Parrott, J. A., et al. Mol Cell Endocrinol 2001 172: 213-222; Wang, J., et al. Int J Cancer 2003 103: 328-334; Zheng, W., et al. Gynecol Oncol 2000 76: 80-88). To confirm the relevance of FSHR as a target, the expression of FSHR was compared in 6 unselected, advanced (stage III-IV) human ovarian carcinomas to that in (sensitive) OVCAR3 cells by Western-blot analysis. Variable expression of the FSH Receptor was identified in most human tumors. In some cases at even higher levels than in OVCAR3 (FIG. 7), supporting the potential of FSH-CERs.

### Example 9: Generation of FOXP1-Ablated, FSHCER-Expressing Human T Cells

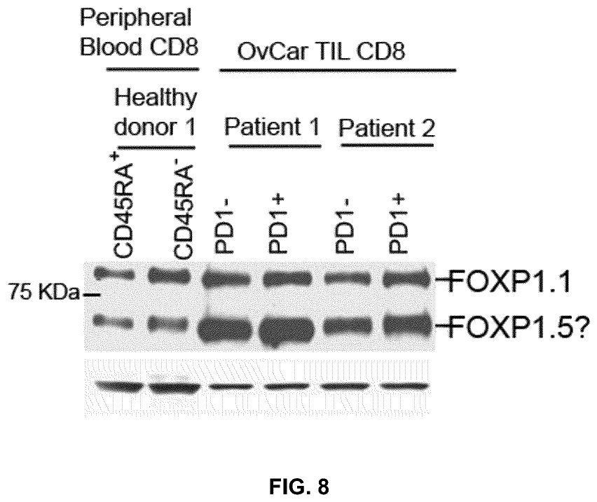

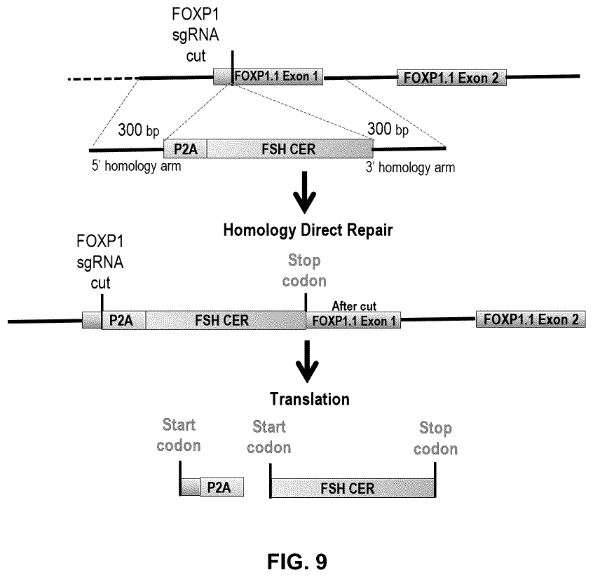

Emerging studies indicate that long (>1 kb) DNA constructs can be effectively integrated into the genome of human T cells while avoiding viral vectors by using CRISPR and homology-directed repair (HDR) (Roth, T. L., et al. Nature 2018 559: 405-409). To circumvent the use of viral vectors and express our FSHCER constructs while targeting FOXP1, a new system was designed to integrate a 1,386 bp DNA cassette immediately after codon #17 encoded by exon 1 of FOXP1.1, which is constitutively expressed in resting and activated human T cells (Stephen, T. L., et al. Immunity 2014 41: 427-439) (FIG. 8). Of note, 6 of the 8 reported splicing variants of FOXP1 share the shame exon 1 and TSS. Based on MW, it was concluded that the shorter variant upregulated in TILs corresponds to FOXP1.5, which would be also ablated with the disclosed system. However, it could theoretically overlap with FOXP1.6. An alternative system to CRISPR at exon 4, which is common to all FOXP1 variants, was therefore designed. The cassette to be integrated encodes all the subunits of the human FSHCER sequence (FIG. 9), downstream of a self-excising 2A peptide. The cellular machinery that drives the constitutive expression of FOXP1 in all T cells will ensure the expression of the FSHCER, while the stop codon at position 1,383 and the relatively long sequence of the construct will prevent the transcription/translation of FOXP1 beyond AA 17 (FIG. 9). Again, FOXP1-deficient T cells do not show any defect in in vitro proliferation and exhibit superior anti-tumor activity at tumor beds. To optimize this system, an oligo was obtained that included a fluorescently labeled tracRNA, a crRNA oligo with the guide specific for FOXP1 (bold), plus the tracrRNA fusion domain: ACGGUUCAGCCAUCCAGAAUGUUUUAGAGCUAGAAA (SEQ ID NO:3). The system has been designed with 300 bp homology arms that will direct integration of the P2A-FSHCER cassette in frame following the ablation of codon #17 of FOXP1.1 at the third base starting from the end of the sequence targeted by the RNA guide. CAS9-based ctRNP complexes are co-electroporated with the HDR template using an Amaxa system and protocols detailed elsewhere (Roth, T. L., et al. Nature 2018 559: 405-409; Osborn, M. J., et al. Mol Ther 2016 24: 570-581; Rahdar, M., et al. Proc Natl Acad Sci USA 2015 112: E7110-7117). Positively electroporated cells are sorted after 24 hrs using the fluorescent label on the tracRNA (550 nm). To verify expression of the FSHCER on the cell surface a FACS analysis is optimized using anti-FSH Abs. As a back-up approach, the expression of FSH is tested by Q-PCR.

### Example 10: Generation of sqRNA

To generate RNP, CAS9 and the sgRNA guide are mixed together. The RNA component of the RNP (sgRNA), encoding the guide RNA can be manufactured using a polymerase chain reaction (PCR)-generated template, followed by in vitro transcription. This template is the DNA sequence of the gRNA of interest obtained from any appropriate source such as plasmid DNA, cDNA, or synthetic DNA sequence.

The template must contain appropriate promoters and a corresponding RNA polymerase. For example, to use the T7 mScript™ RNA system (Catalog no. C-MSC11610, Cellscript, WI, USA) requires the T7 bacteriophage promoter (TAATACGACTCACTATAG (SEQ ID NO:4)) upstream of the double-stranded DNA template. Other RNA production kits using different promoter systems, such as SP6 and T3 are also available and can be used for synthesis of mRNA to be used for this protocol:

For gRNA, the following gblock is ordered and used as PCR template (alternative: GGGACTGAGACAAAAAGTAA (SEQ ID NO:5)):

T7 promoter_target guide RNA (FOXP1; lower case indicates predicted

cleavage)_guide RNA scaffold_Termination signal:

(SEQ ID NO: 6)
TAATACGACTCACTATAG_ACGGTTCAGCCATCCAgAAT_GTTTTAGA
GCTAGAAATAGCAAGTTAAAATAAGGCTAGTCCGTTATCAACTTGAAA
AAGTGGCACCGAGTCGGTGCTTTTTTTCTAGACCCAGCTTTCTTGTAC
AAAGTTGGCATTA.

Primers for PCR are provided in Table 2.

TABLE 2
Primers for PCR:

| OLIGO | start | len | tm | gc % | any | 3′ | seq |
|---|---|---|---|---|---|---|---|
| LEFT PRIMER | 1 | 31 | 63.04 | 35.48 | 8.00 | 2.00 | TAATACGACTCACTATAGACGGTTCAGC (SEQ ID NO: 7) |
| RIGHT PRIMER | 154 | 24 | 57.68 | 33.33 | 8.00 | 5.00 | TAATGCCAACTTTGTACAAGAAAG (SEQ ID NO: 8) |

SEQUENCE SIZE: 154

### Example 11: Generation of RNP Using In Vitro Transcribed sgRNA

Cas9 and gRNA components are combined to form RNP complexes for electroporation. Cas9 mRNA can be directly purchased from Sigma (EC Number 231-791-2; 25 μg: $500); or Clontech (Cat. No. 632641). Nuclease-free water is used in buffers to resuspend or dilute RNA to prevent degradation. The following protocol is used:
1. Bring 100 pmol of Cas9 to a final volume of 5 μL using Cas9 buffer (20 mM HEPES-KOH PH 7.5, 150 mM KCl, 10% glycerol, 1 mM TCEP). From 40 μM stock: 2.5 μL.
2. Bring 120 pmol sgRNA to a final volume of 5 μL using Cas9 buffer. This means you will need a minimum sgRNA concentration of 24 μM.
3. Add Cas9 to sgRNA slowly while swirling pipette tip, should take 30 s to 1 minute. In other words, premix 2 μg of Cas9 protein and 2 μg of sgRNA in 1× Cas9 reaction buffer in a total volume of 10 μl for each shot. The molar ratio of sgRNA to Cas9 protein is kept at ˜4-5:1.
4. Allow RNP to form at room temperature (25° C.) for 10-20 minutes. Excess of the 1 μM RNP complex can be made and stored for later use at −80° C. for at least 3 months.
5—Add 2 μL of 1 μg/μL (4 μg, total) double-stranded or single-stranded oligonucleotide donor DNA (ssODN) encoding the hFSH_CER construct and the 2 recombination arms to the cuvettes.

### Example 11: Generation of RNP Using Synthetic crRNA and tracrRNA

A (fluorescently labeled; 550 nm) tracRNA is ordered from IDT DNA (Alt-R® CRISPR-Cas9 tracrRNA, ATTO™ 550; e.g., Catalog #1075928).

The crRNA (RNA) oligo is ordered from IDT DNA or Sigma, with the following sequence, with the guide specific for FoxP1, plus the 16 nt tracrRNA fusion domain: (or order from pre-designed sequences from IDT DNA: ACGGUUCAGCCAUCCAGAAU-GUUUUAGAGCUAGAAA (SEQ ID NO:9).

Guide RNA complexes are formed by combining the crRNA and tracrRNA in equal molar amounts in IDT Duplex Buffer (30 mM HEPES, pH 7.5, 100 mM Potassium Acetate) at 1 μM concentration by heating the oligos to 95° C. and slowly cooling to room temperature. Keep working stocks of crRNAs and tracrRNA at 10 μM concentration in TE (10 mM Tris, pH 7.5, 0.1 mM EDTA), in which case a mix 1 μL of crRNA and 1 μL of tracrRNA with 8 μL of Duplex Buffer is made.

While not always necessary, the heat/cool step improves performance for approximately 10% of target sites.

Excess of the 1 μM crRNA:tracrRNA complex can be stored for later use at 4° C., −20° C. or −80° C. for at least 3 months.

Alt-R™ 3NLS Cas9 Nuclease (Integrated DNA Technologies) is diluted from stock 61 μM (10 mg/mL) to 1 μM in Opti-MEM (Thermo Fisher Scientific, Carlsbad, CA USA). Final transfections will employ 10 nM ctRNP complex.

The ctRNP complex is prepared by combining 5.25 μL of the 1 μM crRNA:tracrRNA complex with 5.25 μL of the 1 μM diluted stock of Cas9 protein. (Note: excess of the 1 μM RNP complex can be made and stored for later use at 4° C. or −80° C. for at least 3 months.). Add 77 μL of Opti-MEM medium, bringing the final volume to 87.5 μL, yielding a final 60 nM concentration of RNP complex.

This mixture is incubated at room temperature for 5 min.

### Example 12: Components for Electroporation

ECM830 Electro Square Wave Porator (Harvard Apparatus BTX, MA, USA).

2 mm cuvette (Catalog no. 1652086, Biorad, Hercules, CA, USA).

RNA introduction into target cells can be carried out using other available electroporation instruments that are commercially available, including, but not limited to Amaxa Nucleofactor-II (Amaxa Biosystems, Cologne, Germany), Gene Pulser Xcell (Biorad, Denver, CO, USA) or Multiporator (Eppendorf, Hamburg, Germany).

Blazar's protocol uses a Neon Transfection system (Thermo), buffer T 1400V, 10 ms and 3 pulses of electroporation.

### Example 13: Generation of ssDNA HDRT (Try First with dsDNA HDRT)

Double-stranded DNA HDRT production: Order through GenScript the homology arms and the desired hFSH_CER insert. The plasmid is used as template for high-output PCR amplification. PCR amplicons (the dsDNA HDRT) is purified and eluted into a final volume of 3 μl H2O per 100 μl of PCR reaction input. Concentrations of HDRTs are determined by nanodrop using a 1:20 dilution.

Single-stranded DNA HDRT production by exonuclease digestion: To produce long ssDNA as HDR templates, the DNA of interest was amplified via PCR using one regular, non-modified PCR primer and a second phosphorylated PCR primer. The DNA strand that will be amplified using the phosphorylated primer will be the strand that will be degraded using this method. This makes it possible to prepare either a single-stranded sense or single-stranded antisense DNA using the respective phosphorylated PCR primer. To produce the ssDNA strand of interest, the phosphorylated strand of the PCR product was degraded by treatment with two enzymes, Strandase Mix A and Strandase Mix B, for 5 min (per 1 kb) at 37° C., respectively. Enzymes were deactivated by a 5 min incubation at 80° C. The resulting ssDNA HDR templates are purified and eluted in H₂O. A more detailed protocol for the Guide-it Long ssDNA Production System (Takara Bio, 632644) can be found at the manufacturer's website.

### Example 14: Electroporation Method

Wash T cells to be electroporated (×3 times) using Opti-MEM I: Reduced serum medium (Catalog no. 31985, Gibco, Grand Island, NY, USA). Centrifuge cells at 300 x× g for 5 minutes at 4° C.

Carefully discard supernatant and resuspend cell pellet in fresh Opti-MEM media at 1×10⁸ cells/ml. For each electroporation, aliquot 1×10⁷ cells in a 100 μl of Opti-MEM. Keep cells on ice until use.

Pre-configure the electroporator by setting the voltage to 500V and time to 1000 μ-seconds.

Prewarm R10 media to 37° C. and add 10 ml of the media to a T25 flask. Place the cells in 100 μl into a 2 mm cuvette.

Add 10 μL of RNP complex plus 2 μL HDRT with the 100 μl aliquot of cells. Uniformly mix by gentle pipetting.

Place the cuvette into the electroporator cassette, tighten the electrodes around the metal plates of the cuvette and initiate the electric pulse.

Immediately transfer the contents of the cuvette into the T25 flask containing R10.

Rinse the cuvette once with fresh R10 to maximize recovery of electroporated cells.

Place the cells in a 37° C. CO2 incubator until further use.

After 25 hrs, positively electroporated cells can be sorted by using the fluorescent label on the tracRNA (550 nm), after 24 hrs. To verify expression of the CER on the cell surface FACS analysis using anti-FSH Abs can be used, or the expression of FSH can be tested by Q-PCR using the following primers:

TABLE 3
Primers for Q-PCR:

| OLIGO | start | len | tm | gc % | any th | 3′ th | seq |
|---|---|---|---|---|---|---|---|
| LEFT PRIMER | 73 | 23 | 61.73 | 47.83 | 0.00 | 0.00 | CTGACCAACATCACAATCGCCAT (SEQ ID NO: 10) |
| RIGHT PRIMER | 209 | 23 | 61.68 | 52.17 | 0.00 | 0.00 | GTTTTCTGGATCTTGGGTCTGGC (SEQ ID NO: 11) |

SEQUENCE SIZE: 137

or

TABLE 4
Primers for Q-PCR:

| Sequence (5′ → 3′) | Template strand | Tm |
|---|---|---|
| Forward primer | ACAGAGTGACCGTGATGGGC | 54 |
| | (SEQ ID NO: 12) | |
| Reverse primer | CTGGGAGGCGATTGTAGGGG | 155 |
| | (SEQ ID NO: 13) | |

CRISPRed T cells are then expanded against irradiated K562 cells transduced with human FSHR (1:10 ratio), in R10 media with II-2 (300 UI/mL).

HDRT sequence (FOXP1; insert is 1386 bp+P2A)

5′-arm:
(SEQ ID NO: 14)
GTAATATATTTAAAACAATACTACTGAAACTTGAATTGGGAATGACAGTT
TCAGACCCGAAACTAGGGGCATGGCCCACTAATGAGGGGATAAGTTGAGT
GAAAGAAAATGACAACTGTTTACAGATTTTGGCAACATTTAAACCAAGGC
CCTTCTCCTTATGCACAACAACTGCTTTAACAGCTGCTTTTTTTTTCTCC
CCCCCTCCCTCCCCCCATCTTGGAAATCCTTGTATCAGGTTTTTGAGTCA
TGATGCAAGAATCTGGGACTGAGACAAAAAGTAACGGTTCAGCCATCCA
G;
P2A:
(SEQ ID NO: 15)
gccacgaacttctccctgttaaagCaagcaggagacgtggaagaaaaccc
cggtccc

Insert (at place of CRISPR excision (CCAG AAT)):

(SEQ ID NO: 16)
ATGAAGACCCTGCAGTTCTTCTTCCTGTTCTGCTGCTGGAAGGCCATCTG
CTGCAACAGCTGCGAGCTGACCAACATCACAATCGCCATCGAGAAAGAGG
AATGCCGGTTCTGCATCAGCATCAACACCACTTGGTGCGCCGGCTACTGC
TACACCCGGGACCTGGTGTACAAGGACCCCGCCAGACCCAAGATCCAGAA
AACCTGCACCTTCAAAGAACTGGTGTACGAGACAGTGCGGGTGCCCGGAT
GTGCCCACCATGCCGATAGCCTGTACACCTACCCTGTGGCCACCCAGTGT
CACTGCGGCAAGTGCGATAGCGACAGCACCGATTGCACCGTGCGGGGACT
GGGCCCTAGCTACTGTAGCTTCGGCGAGATGAAGGAAGGCGGCGGATCTG
GCGGAGGAAGCGGAGGGGGATCTGGGGGCGGAGCACCTGATGTGCAGGAT
TGCCCTGAGTGCACCCTGCAGGAAAACCCATTCTTCAGCCAGCCTGGCGC
CCCTATCCTGCAGTGCATGGGCTGCTGCTTCAGCAGAGCCTACCCCACCC
CCCTGCGGAGCAAGAAAACCATGCTGGTGCAGAAAAACGTGACCAGCGAG
AGCACCTGTTGCGTGGCCAAGAGCTACAACAGAGTGACCGTGATGGGCGG
CTTCAAGGTGGAAAACCACACCGCCTGCCACTGCAGCACATGCTACTACC
ACAAGAGCGCTAGCACCACCACCCCTGCCCCTAGACCTCCAACACCCGCC
CCTACAATCGCCTCCCAGCCTCTGTCTCTGAGGCCCGAGGCTTGTAGACC
AGCTGCTGGCGGAGCCGTGCACACCAGAGGACTGGATTTCGCCTGCGACA
TCTACATCTGGGCCCCTCTGGCCGGCACATGTGGCGTGCTGCTGCTGAGC
CTCGTGATCACCCTGTACTGCAAGCGGGGCAGAAAGAAGCTGCTGTACAT
CTTCAAGCAGCCCTTCATGCGGCCCGTGCAGACCACCCAGGAAGAGGACG
GCTGCTCCTGCAGATTCCCCGAAGAGGAAGAGGGGGGCTGCGAACTGAGA
GTGAAGTTCAGCAGAAGCGCCGACGCCCCTGCCTACAAGCAGGGCCAGAA
CCAGCTGTACAACGAGCTGAACCTGGGCAGACGGGAAGAGTACGACGTGC
TGGACAAGCGGAGAGGCAGGGACCCTGAGATGGGCGGAAAGCCCAGACGG
AAGAACCCCCAGGAAGGCCTGTATAACGAACTGCAGAAAGACAAGATGGC
CGAGGCCTACAGCGAGATCGGAATGAAGGGCGAGCGGAGAAGAGGCAAGG
GCCACGATGGCCTGTACCAGGGCCTGAGCACCGCCACCAAGGACACCTAT
GACGCCCTGCACATGCAGGCCCTGCCCCCCAGATAA;
3′-arm:
(SEQ ID NO: 17)
AATGGGTCGGGCGGCAGCAACCACTTACTAGAGTGCGGCGGTCTTCGGGA
GGGGCGGTCCAACGGAGAGACGCCGGCCGTGGACATCGGGGCAGCTGACC
TCGCCCACGCCCAGCAGCAGCAGCAACAGGTACTGGGCTTTGGAGTCTTG
GGAGGTGGAGGTGGTGGGTGCGAATTTTACTGCTGAATTTTACACTTAAT
CGCGATCTCGCGATCAGTGTTTTGTGTTCACCTGTCTGTCCATCTGTCTT
TCTGTCTGTCAGTGGCATCTCATAAACCATCAGCCCTCTAGGAGTCCCA.

Foxp1_P2A_SP of CER_hFSHbeta_linker_CGalpha_Hinge (from CD8)_TM domain (from hCD8)_4-1BB (intracellular)_human CD3zeta domain:

(SEQ ID NO: 18)
ATGATGCAAGAATCTGGGACTGAGACAAAAAGTAACGGTTCAGCCATCCAG_gccacga
acttctccctgttaaagCaagcaggagacgtggaagaaaaccccggtccc_ATGAAGACCCTGCAGTTCTTCT
TCCTGTTCTGCTGCTGGAAGGCCATCTGCTGC_AACAGCTGCGAGCTGACCAACATCA
CAATCGCCATCGAGAAAGAGGAATGCCGGTTCTGCATCAGCATCAACACCACTTGGTG
CGCCGGCTACTGCTACACCCGGGACCTGGTGTACAAGGACCCCGCCAGACCCAAGAT
CCAGAAAACCTGCACCTTCAAAGAACTGGTGTACGAGACAGTGCGGGTGCCCGGATGT
GCCCACCATGCCGATAGCCTGTACACCTACCCTGTGGCCACCCAGTGTCACTGCGGCA
AGTGCGATAGCGACAGCACCGATTGCACCGTGCGGGGACTGGGCCCTAGCTACTGTA
GCTTCGGCGAGATGAAGGAA_GGGGGGGGATCTGGCGGAGGAAGCGGAGGGGGATC
TGGGGGCGGA_GCACCTGATGTGCAGGATTGCCCTGAGTGCACCCTGCAGGAAAAC
CCATTCTTCAGCCAGCCTGGCGCCCCTATCCTGCAGTGCATGGGCTGCTGCTTCAGC
AGAGCCTACCCCACCCCCCTGCGGAGCAAGAAAACCATGCTGGTGCAGAAAAACGT
GACCAGCGAGAGCACCTGTTGCGTGGCCAAGAGCTACAACAGAGTGACCGTGATGG
GCGGCTTCAAGGTGGAAAACCACACCGCCTGCCACTGCAGCACATGCTACTACCACA
AGAGC_GCTAGCACCACCACCCCTGCCCCTAGACCTCCAACACCCGCCCCTACAATCG
CCTCCCAGCCTCTGTCTCTGAGGCCCGAGGCTTGTAGACCAGCTGCTGGCGGAGCCG
TGCACACCAGAGGACTGGATTTCGCCTGC_GACATCTACATCTGGGCCCCTCTGGCCG
GCACATGTGGCGTGCTGCTGCTGAGCCTCGTGATCACCCTGTACTGCAAGCGGGGCA
GAAAGAAGCTGCTGTACATCTTC_AAGCAGCCCTTCATGCGGCCCGTGCAGACCACCC
AGGAAGAGGACGGCTGCTCCTGCAGATTCCCCGAAGAGGAAGAGGGGGGCTGCGAAC
TG_AGAGTGAAGTTCAGCAGAAGCGCCGACGCCCCTGCCTACAAGCAGGGCCAGAA
CCAGCTGTACAACGAGCTGAACCTGGGCAGACGGGAAGAGTACGACGTGCTGGACA
AGCGGAGAGGCAGGGACCCTGAGATGGGCGGAAAGCCCAGACGGAAGAACCCCCA
GGAAGGCCTGTATAACGAACTGCAGAAAGACAAGATGGCCGAGGCCTACAGCGAGA
TCGGAATGAAGGGCGAGCGGAGAAGAGGCAAGGGCCACGATGGCCTGTACCAGGG
CCTGAGCACCGCCACCAAGGACACCTATGACGCCCTGCACATGCAGGCCCTGCCCC
CCAGA_TAA;
(SEQ ID NO: 19)
MMQESGTETKSNGSAIQ_ATNFSLLKQAGDVEENPGP_MKTLQFFFLFCCWKAICC_NSC
ELTNITIAIEKEECRFCISINTTWCAGYCYTRDLVYKDPARPKIQKTCTFKELVYETVRVPGCA
HHADSLYTYPVATQCHCGKCDSDSTDCTVRGLGPSYCSFGEMKE_GGGSGGGSGGGSG
GG_APDVQDCPECTLQENPFFSQPGAPILQCMGCCFSRAYPTPLRSKKTMLVQKNVTSE
STCCVAKSYNRVTVMGGFKVENHTACHCSTCYYHKS_ASTTTPAPRPPTPAPTIASQPLSL
RPEACRPAAGGAVHTRGLDFAC_DIYIWAPLAGTCGVLLLSLVITLYC_KRGRKKLLYIFKQ
PFMRPVQTTQEEDGCSCRFPEEEEGGCEL_RVKFSRSADAPAYKQGQNQLYNELNLGRR
EEYDVLDKRRGRDPEMGGKPRRKNPQEGLYNELQKDKMAEAYSEIGMKGERRRGKGH
DGLYQGLSTATKDTYDALHMQALPPR.

HDRT sequence to be ordered (2043 bp):

(SEQ ID NO: 20)
GTAATATATTTAAAACAATACTACTGAAACTTGAATTGGGAATGACAGTT
TCAGACCCGAAACTAGGGGCATGGCCCACTAATGAGGGGATAAGTTGAGT
GAAAGAAAATGACAACTGTTTACAGATTTTGGCAACATTTAAACCAAGGC
CCTTCTCCTTATGCACAACAACTGCTTTAACAGCTGCTTTTTTTTTCTCC
CCCCCTCCCTCCCCCCATCTTGGAAATCCTTGTATCAGGTTTTTGAGTCA
TGATGCAAGAATCTGGGACTGAGACAAAAAGTAACGGTTCAGCCATCCAG
gccacgaacttctccctgttaaagCaagcaggagacgtggaagaaaaccc
cggtcccATGAAGACCCTGCAGTTCTTCTTCCTGTTCTGCTGCTGGAAGG
CCATCTGCTGCAACAGCTGCGAGCTGACCAACATCACAATCGCCATCGAG
AAAGAGGAATGCCGGTTCTGCATCAGCATCAACACCACTTGGTGCGCCGG
CTACTGCTACACCCGGGACCTGGTGTACAAGGACCCCGCCAGACCCAAGA
TCCAGAAAACCTGCACCTTCAAAGAACTGGTGTACGAGACAGTGCGGGTG
CCCGGATGTGCCCACCATGCCGATAGCCTGTACACCTACCCTGTGGCCAC
CCAGTGTCACTGCGGCAAGTGCGATAGCGACAGCACCGATTGCACCGTGC
GGGGACTGGGCCCTAGCTACTGTAGCTTCGGCGAGATGAAGGAAGGCGGC
GGATCTGGCGGAGGAAGCGGAGGGGGATCTGGGGGCGGAGCACCTGATGT
GCAGGATTGCCCTGAGTGCACCCTGCAGGAAAACCCATTCTTCAGCCAGC
CTGGCGCCCCTATCCTGCAGTGCATGGGCTGCTGCTTCAGCAGAGCCTAC
CCCACCCCCCTGCGGAGCAAGAAAACCATGCTGGTGCAGAAAAACGTGAC
CAGCGAGAGCACCTGTTGCGTGGCCAAGAGCTACAACAGAGTGACCGTGA
TGGGCGGCTTCAAGGTGGAAAACCACACCGCCTGCCACTGCAGCACATGC
TACTACCACAAGAGCGCTAGCACCACCACCCCTGCCCCTAGACCTCCAAC
ACCCGCCCCTACAATCGCCTCCCAGCCTCTGTCTCTGAGGCCCGAGGCTT
GTAGACCAGCTGCTGGCGGAGCCGTGCACACCAGAGGACTGGATTTCGCC
TGCGACATCTACATCTGGGCCCCTCTGGCCGGCACATGTGGCGTGCTGCT
GCTGAGCCTCGTGATCACCCTGTACTGCAAGCGGGGCAGAAAGAAGCTGC
TGTACATCTTCAAGCAGCCCTTCATGCGGCCCGTGCAGACCACCCAGGAA
GAGGACGGCTGCTCCTGCAGATTCCCCGAAGAGGAAGAGGGGGGCTGCGA
ACTGAGAGTGAAGTTCAGCAGAAGCGCCGACGCCCCTGCCTACAAGCAGG
GCCAGAACCAGCTGTACAACGAGCTGAACCTGGGCAGACGGGAAGAGTAC
GACGTGCTGGACAAGCGGAGAGGCAGGGACCCTGAGATGGGCGGAAAGCC
CAGACGGAAGAACCCCCAGGAAGGCCTGTATAACGAACTGCAGAAAGACA
AGATGGCCGAGGCCTACAGCGAGATCGGAATGAAGGGCGAGCGGAGAAGA
GGCAAGGGCCACGATGGCCTGTACCAGGGCCTGAGCACCGCCACCAAGGA
CACCTATGACGCCCTGCACATGCAGGCCCTGCCCCCCAGATAAAATGGGT
CGGGCGGCAGCAACCACTTACTAGAGTGCGGCGGTCTTCGGGAGGGGCGG
TCCAACGGAGAGACGCCGGCCGTGGACATCGGGGCAGCTGACCTCGCCCA
CGCCCAGCAGCAGCAGCAACAGGTACTGGGCTTTGGAGTCTTGGGAGGTG
GAGGTGGTGGGTGCGAATTTTACTGCTGAATTTTACACTTAATCGCGATC
TCGCGATCAGTGTTTTGTGTTCACCTGTCTGTCCATCTGTCTTTCTGTCT
GTCAGTGGCATCTCATAAACCATCAGCCCTCTAGGAGTCCCAG.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A chimeric cell expressing a chimeric receptor,
wherein the chimeric receptor is encoded by a transgene, and
wherein the transgene is inserted at a loci within a Foxp1 gene in a genome of a cell thereby disrupting expression or activity of an endogenous Foxp1 protein.

2. The chimeric cell of claim 1, wherein the chimeric receptor comprises two subunits of a follicule-stimulating hormone (FSH).

3. The chimeric cell of claim 1, wherein the chimeric receptor is a chimeric antigen receptor (CAR) polypeptide.

4. The chimeric cell of claim 1, wherein the cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell.

5. A method of providing an anti-cancer immunity in a subject, comprising administering to the subject an effective amount of the chimeric cell of claim 1, thereby providing an anti-tumor immunity in the subject.

6. The method of claim 5, wherein the chimeric receptor comprises two subunits of a follicule-stimulating hormone (FSH), and wherein the subject has an FSH-receptor positive ovarian tumor.

7. The method of claim 5, further comprising administering to the subject a checkpoint inhibitor.

8. The method of claim 7, wherein the checkpoint inhibitor comprises an anti-programmed cell death protein 1 (PD-1) antibody, anti-programmed death-ligand 1 (PD-L1) antibody, anti-cytotoxic T-lymphocyte-associated antigen 4 (CTLA-4) antibody, or a combination thereof.
